SEQUENCE LISTING (2 sequences)

SEQ ID NO: 1 - DNA, 22 bp, Artificial
  misc_feature (1)..(22): Analyte sequence
gtcccctacggacaaggcgcgt

SEQ ID NO: 2 - DNA, 27 bp, Artificial
  misc_feature (1)..(27): Control sequence
tttttcgcgccttgtccgtaggggact

(19) United States
(12) Patent Application Publication
(10) Pub. No.: US 2004/0157263 A1
(43) Pub. Date: Aug. 12, 2004

(54) Method for impedimetric detection of one or more analytes in a sample, and device for use therin

(75) Inventors: Edgar Diessel, Koln (DE); Werner Hoheisel, Koln (DE); Udo Merker, Koln (DE); Jens Burmeister, Koln (DE); Burkhard Kohler, Leverkusen (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Bayer Aktiengesellschaft,, Leverkusen (DE)

(21) Appl. No.: 10/606,301

(22) Filed: Jun. 25, 2003

(30) Foreign Application Priority Data
Jun. 25, 2002 (DE) 102 28 260.9

(51) Int. Cl. C12Q 1/68; G01N 33/53

(52) U.S. Cl. 435/7.1; 205/777.5; 435/6

(57) ABSTRACT

Method for detecting at least one analyte, and device for performing the method comprising a measurement electrode having a biofunctional surface having recognition elements for the analyte, and one or more counterelectrodes. Analyte labeled with electrically active labeling units is brought into contact with the biofunctional surface. Either (a) a time-varying voltage or (b) a time-varying current is applied between a first counterelectrode and the measurement electrode. A measurement is made of either in case (a) the current or in case (b) the voltage between the first counterelectrode and the measurement electrode. Alternatively, a measurement is made of either in case (a) the current or in case (b) the voltage between a second or subsequent counterelectrode and the measurement electrode. This abstract is submitted with the understanding that it will not be used to interpret or limit the meaning or scope of the claims. 37 CFR § 1.72(b).

The invention relates to a method for the qualitative and/or quantitative impedimetric detection of analytes in a sample, and to a device for practicing the method. The method advantageously involves the specific detection of a biologically relevant molecule in an aqueous medium. Such a sensor principle, or such a sensor, has a wide range of application, for example, in environmental analysis, the food industry, human and veterinary diagnosis, crop protection and in biochemical or pharmacological research.

For such diagnostic applications, bio- or chemosensors are known which have a biofunctional surface and a physical signal transducer.

Biological, chemical or biochemical recognition elements, for example, DNA, RNA, aptamers, receptors, to which an analyte binds specifically by means of a recognition reaction during detection, are bound to biofunctional surfaces.

Examples of recognition reactions are the binding of ligands to complexes, the sequestration of ions, the binding of ligands to (biological) receptors, membrane receptors or ion channels, of antigens or haptens to antibodies (immunoassays), of substrates to enzymes, of DNA or RNA to specific proteins, of aptamers or “spiegelmers” to their targets, the hybridization of DNA/RNA/PNA or other nucleic acid analogues (DNA assays), or the processing of substrates by enzymes.

Examples of analytes to be detected are DNA, RNA, PNA, nucleic acid analogues, enzyme substrates, peptides, proteins, potential active agents, medicaments, cells, or viruses.

Examples of recognition elements, to which the analytes to be detected bind, are DNA, RNA, PNA, nucleic acid analogues, aptamers, “spiegelmers”, peptides, proteins, sequestrants for metals/metal ions, cyclodextrins, crown ethers, antibodies or fragments thereof, anticalins, enzymes, receptors, membrane receptors, ion channels, cell adhesion proteins, gangliosides, or mono- or oligosaccharides.

Recognition elements can be coupled covalently or non-covalently to the biofunctional surface. Covalent immobilization of recognition elements, for example, DNA, on sensor surfaces has decisive advantages, in terms of stability, reproducibility and specificity of the coupling, over non-covalent coupling. A review of methods for preparing DNA-coated surfaces is given by S. L. Beaucage, Curr. Med., 2001, 8, 1213-1244.

An example of non-covalent coupling is the spotting of cDNA on glass supports, on which polylysine has been adsorbed beforehand.

If a variety of recognition elements are bound to the surface of the signal transducer so that they are spatially separated from one another, then a large number of recognition reactions can be carried out simultaneously with a sample to be studied. This is done, for example, in so-called DNA arrays, in which various DNA sequences (for example, oligonucleotides or cDNAs) are immobilized on a solid support (for example, glass). Such DNA arrays are generally read by using optical methods, or alternatively by using electrical methods, and they are employed in expression profiling, sequencing, detection of viral or bacterial nucleic acids, genotyping, etc.

The recognition reaction in bio- or chemosensors may be detected by using optical, electrical/electrochemical, mechanical and magnetic signal transduction methods.

Although the most advanced described optical methods, in particular, have high sensitivities, they can generally be miniaturized only to a limited extent because of the complex structure involving a light source, sensor and photodetector, and they therefore remain inferior to electrical methods with respect to production costs.

For this reason, increased importance is being attached to the development of electrical sensors. In particular, the use of microstructuring techniques from semiconductor technology leads to miniaturized formats which offer high sensitivities. DE 43 185 19 and WO 97/21094 use microstructured electrode arrangements in order to detect specific binding of unlabeled antibodies or DNA to antigens or complementary DNA, which are immobilized between two electrodes, by means of impedance measurements. In particular, the molecules to be detected are labeled with reversibly reducible or oxidizable molecules in DE-A 4 318 519, so that amplification effects are achieved by electrochemical recycling in these interdigitated structures.

An alternative method of amplifying electrochemical signals involves enzyme-induced precipitation of polymers, which significantly increase the electron transfer resistance (Patolsky et al., Langmuir 15, 3703 (1999)).

Electrochemical methods can be compromised by unspecific detection of electro-active substances such as are present in real samples, for example, bodily fluids.

Field-effect transistors are used for the detection of charged molecules, or complexes of charged molecules and ligands (U.S. Pat. No. 6,203,981).

Nanoparticles can be used as an alternative substrate material. In EP-A 1 022 560 A1, the conductivity of a nanoparticle network is modified by ligand adsorption. WO 01/13432 A1 discloses the use of an individual nanoparticle as a single-electron transistor, the current-voltage characteristic of which is influenced by ligand adsorption.

In these concepts, which are based on electrostatic field effects, interference effects between the targeted ligand adsorption and unspecific adsorptions of charged molecules onto the sensor surface can occur in real samples, for example, blood, or urine.

Methods which use labeling units for the analytes, the properties of which differ significantly from those of the constituents of the sample to be analyzed, are superior in this regard. To that end, for example, metallic nanoparticles are suitable as labeling units.

U.S. Pat. No. 5,858,666 discloses the use of metallic nanoparticles as labeling units in electrical biosensor technology. In the scope of DC measurements, certain electrical biosensors with metallic nanoparticles have the potential for extraordinarily high sensitivity, down to the single-molecule range. This potential is facilitated, in particular, by autometallographic deposition. In this so-called autometallography process, which is known from photography and electron microscopy, the nanoparticles or colloids act as catalysts for the electron transfer from a reducing agent to an Au or Ag ion, which the amplification solution contains in the form of an Ag or Au salt with the reducing agent, for example, hydroquinone. After reaction has taken place, the ion precipitates as metal onto the colloid. Electrode pairs, which are separated from one another by an insulator, are to that end selected as the electrical signal transducer. With autometallographic enlargement, analyte molecules labeled with nanoparticles form a conductive bridge between the electrodes, and this is detected by a DC resistance measurement. The fundamental patents for this are U.S. Pat. No. 4,794,089; U.S. Pat. No. 5,137,827; U.S. Pat. No. 5,284,748. Further disclosures can be found in DE-A 198 60 547, WO 99/57550 and in WO 01/00876. The detection of nucleic acids by DC resistance measurement has been demonstrated (Moller et al., Langmuir, 17, 5426 (2001)). As a further development stage of this method, the discrimination of point mutations (single nucleotide polymorphisms (SNPs)) is described in Park et al., Science, 295, 1503 (2002).

In the latter two embodiments, the electrode spacings are very much larger than the particles after the autometallography process (a factor of about 100-1000). A percolation path therefore needs to be formed between the electrodes, in order to permit a flow of current. This restricts the dynamic range of the measurement method very significantly, so that these methods are generally used only as threshold-value methods. Dynamic ranges are facilitated only by a very elaborate multiple autometallographic enlargement process, which is not recommendable for practical use in a biosensor.

It is an object of the invention to develop a highly sensitive electrical sensor and a measurement method for the detection of analytes by means of recognition reactions, which have a high sensitivity and can also be quantified in respect of the amount of analytes to be detected.

The object of the invention is achieved by a method for detecting one or more analytes by using a recognition reaction, with the following steps

(a) providing a device with

(i) a measurement electrode with a biofunctional surface, the biofunctional surface having recognition elements for the analytes,

(ii) one or more counterelectrodes,

(iii) a liquid electrolyte between the measurement electrode and the counterelectrodes,

(b) bringing analytes labeled with electrically active labeling units into contact with the biofunctional surface, the electrically active labeling units either having been bound to the analytes before contact of the analytes with the biofunctional surface or being bound to the analytes after contact of the analytes with the biofunctional surface,

(c) applying (i) a time-varying voltage or (ii) a time-varying current between the first counterelectrode and the measurement electrode, and

(d1) either in case (c)(i) measuring the current or in case (c)(ii) measuring the voltage between the first counterelectrode and the measurement electrode, or

(d2) in case (c)(i) measuring the current or in case (c)(ii) measuring the voltage between the second or another counterelectrode and the measurement electrode.

According to the invention, recognition elements for the analytes are bound to the measurement electrode with a biofunctional surface. The analytes enter into a recognition reaction with the recognition elements.

A time-varying voltage or a time-varying current is applied between the measurement electrode and a counterelectrode. The time-varying voltage may, for example, be an AC voltage or a pulsed voltage, and the time-varying current may, for example, be an alternating current or a pulsed current.

When the time-varying voltage or the time-varying current is applied, a Helmholtz double layer with a particular impedance is formed at the electrodes. The impedance of this Helmholtz double layer is modified when analytes which are labeled with an electrically active labeling unit become bound to the biofunctional surface by the recognition reaction, for example, since the area of the measurement electrode is increased by the electrically active labeling units, in particular, by electrical contact between conductive labeling units and the measurement electrode.

The analyte may already be labeled with an electrically active labeling unit before the binding to the recognition element, or alternatively it is not labeled until after the binding to the recognition element, for example, as a result of a binding element, which is labeled with a labeling unit, becoming bound to the complex consisting of the recognition element and the molecule.

With the method according to the invention, it is possible to detect the modification of the impedance due to a single labeling unit, that is to say in general due to a single labeled analyte. Each labeling unit contributes to a measurement signal independently of other labeling units. Analyte molecules may furthermore be provided with a plurality of labeling units, in order to increase the sensitivity of the method even further.

The recognition elements are immobilized on the surface of the measurement electrode by prior-art methods which are known to the person skilled in the art. For DNA recognition units, this immobilization is described, for example, in S. L. Beaucage, Curr. Med., 2001, 8, 1213-1244.

For the immobilization on the electrode surface, it is desirable to have an optimum density of recognition units which, with a high surface density, ensures optimum activity of the recognition unit.

The recognition elements, such as antibodies, may be immobilized covalently or non-covalently. For example, avidin or streptavidin may be physisorbed onto the surface or covalently immobilized after suitable biofunctionalization of the surface. Biotinylated antibodies, for example, can be specifically immobilized onto the surface coated with avidin or streptavidin.

The capacitance of the double layer can be computationally derived from the impedance measurements by using suitable equivalent circuit diagrams.

In order to adjust the working point of the impedance measurement, a DC voltage or a direct current may be superimposed on the time-varying voltage or the time-varying current, respectively.

The method according to the invention can be used, for example, in an immunoassay or a DNA assay. DNA assays are preferably used for detecting viral DNA or RNA, or DNA of bacterial species, as well as expression profiling, genotyping for the diagnosis of hereditary diseases or for pharmacogenomics (genetically related activity or side-effects of pharmaceuticals), nutrigenomics (genetically related activity or side-effects of foodstuffs). In particular, modifications of genes which are due to the variation of only one base (single nucleotide polymorphism=SNP) are established in genotyping.

The analytes may also be detected indirectly by using the recognition reaction. In the case of indirect detection, analytes which are already labeled with labeling units before binding to the recognition element are brought into contact with the biofunctional surface. At the same time, unlabeled analytes are also brought into contact with the biofunctional surface. These two species compete in respect of binding to the immobilized recognition elements. If there are no unlabeled analytes in the electrolyte between the measurement electrode and the counterelectrode, then all the binding sites on the recognition elements will be occupied by labeled analytes, and the modification of the impedance will be a maximum. In the event of a non-zero concentration of unlabeled analytes, some of the binding sites on the recognition elements will be occupied by unlabeled analytes, and some will be occupied by labeled analytes, according to the concentrations in question, so that the modification of the impedance is smaller compared with when the concentration of the unlabeled analyte is zero.

In the method according to the invention, analytes are labeled with labeling units which are active electrically.

The electrical activity may consist of the electrical conductivity of the material used for the labeling units, which is preferably in the range of metallic conductivities.

Nanoparticles, metal complexes and/or clusters of conductive materials such as Au, Ag, Pt, Pd, Cu or carbon may be used as the electrically active labeling units.

The electrical activity may, however, also consist of the dielectric property of the material used for the labeling units. The dielectric constant of the labeling unit is advantageously in the range of from 5 to 15,000, particularly preferably in the range of between 10 and 1,500.

The size of the electrically active labeling units is preferably in the range of between 1 and 100 nm, preferably in the range of between 1 and 30 nm, and particularly preferably 1-2 nm. Au clusters consisting of 50-150 atoms, with a size in the range of 1-2 nm, are more particularly preferred. The indicated size refers in this case to the largest diameter of the labeling units.

Labeling units with high dielectric constants may be nanoparticles or clusters made of titanates, materials which crystallize in a perovskite lattice, TiO₂ or lead compounds. These often have a size in the range of from 1 to 100 nm. For example, PbSO₄ reaches a dielectric constant of 14 at 100 MHz, and BaTiO₃ reaches a dielectric constant of 3,600 at 100 kHz. The respective frequency dependencies should be taken into account when making a comparison.

Carbon “nanotubes”, nonconductive particles with a conductive coating or nonconductive particles with a metallic coating may furthermore be used as the labeling units. The nonconductive particles may, for example, be polystyrene beads. The conductivity properties can be adjusted in a controlled way in the case of carbon “nanotubes”.

The labeling units may also consist of conductive polymers such as polyanilines, polythiophenes, especially polyethylene dioxythiophene, polyphenylenes, polyphenylene vinylene, polythiophene vinylene, or polypyrroles.

Enzymes, for example, horseradish peroxidase (HRP), may also be used as a labeling unit. HRP induces the polymerization of monomers (the substrate) of electrically conductive polymers, for example, polyaniline.

A further use of HRP according to the invention is the deposition of a polymer to which, for example, nanoparticles of all the labeling units described above are bound directly or indirectly via biotin-streptavidin, biotin-avidin or biotin-NeutrAvidin TM (NeutrAvidin TM, Manufacturer: Pierce Biotechnology, Rockford, Ill., U.S.A.). For the indirect case, the polymer is biotinylated. This principle is referred to as catalyzed reporter deposition (CARD).

Suitable enlargement of the labeling units, as can be achieved, for example, by autometallographic enlargement of metal colloids such as Ag or Au, is particularly advantageous for achieving high sensitivities.

The detection of the analyte is carried out in an aqueous medium as the electrolyte. Bodily fluids such as blood, urine, interstitial fluid and tear fluid are preferred as the aqueous medium.

The invention furthermore relates to a device for detecting one or more analytes using a recognition reaction, comprising

(a) at least one measurement electrode with a biofunctional surface, the biofunctional surface having recognition elements for the analytes,

(b) one or more counterelectrodes,

(c) a liquid electrolyte between the measurement electrode and the counterelectrodes,

(d) analytes, which are labeled with electrically active labeling units and can be brought in contact with the recognition elements of the biofunctional surface,

(e) either (i) a voltage source for applying a time-varying voltage or (ii) a current source for applying a time-varying current between the first counterelectrode and the measurement electrode, and

(f) a measuring instrument for

(i) measuring in case (e)(i) the current or in case (e)(ii) the voltage between the first counterelectrode and the measurement electrode, or

(ii) measuring in case (e)(i) the current or in case (e)(ii) the voltage between the second or another counterelectrode and the measurement electrode.

In the device according to the invention, the measurement electrode, counterelectrodes, electrolytes, recognition elements, analytes and the electrically active labeling units preferably have the properties described in relation to the method.

The surface of the measurement electrode may be divided into a plurality of conductive regions.

Electrodes according to the invention may be configured as planar or in a non-planar geometry.

High sensitivities, down to the single-molecule range, are offered by impedimetric measurements based on microelectrodes with areas in the range of from 1 to 20×1 to 20 μm², preferably from 5 to 15×5 to 15 μm², particularly preferably of 10×10 μm², in which the individual labeling units, for example autometallographically enlarged Au colloids, lead to increases in the electrode areas of the order of a few percent. With individual electrode areas of, for example, 10×10 μm², it is possible to fit 10⁶ elements on a chip with a size of 10×10 μm². These size indications are merely exemplary in nature, and do not preclude other sizes and numbers.

One type of recognition element may be immobilized in each conductive region, or the same type of recognition elements may be immobilized in a plurality of conductive regions.

In order to cover dynamic ranges which extend over an expected quantification range of two to three orders of magnitude of an impedimetric measurement with a single electrode, use is made of electrode areas with various sizes, which differ in their area proportionately to the concentration ranges to be detected. In this case, a plurality of conductive regions, which respectively differ in their size by a factor, preferably by a factor in the range of from 5 to 15, particularly preferably from 9 to 11, are in each case used for one type of recognition unit.

In the planar configuration, there are one or more electrodes laterally next to one another on a substrate. Analyte solutions can be delivered to the electrical sensor arrays via microchannels, which can be etched into the structures. Alternatively, a component provided with microchannels may be used as a cover for a planar substrate.

One example of non-planar geometries is a substrate into which channels are etched vertically, for example, by using a dry-etching method. The walls of these microchannels are covered with electrodes. In these microfluidic channels, the analyte solutions can be brought into the immediate vicinity of the electrodes, so that the response time of the device is shortened, i.e., its sensitivity is increased, owing to reduced diffusion paths/times of the analyte molecules.

Particularly advantageously, a plurality of electrodes are configured laterally next to one another or vertically above one another in the form of layer structures.

Advantageously, the counterelectrode may be fitted on the same substrate as the measurement electrodes, for example, for 2-point impedance measurements. As well as the measurement electrode and the counterelectrode, an additional reference electrode may likewise be fitted on the same substrate for a 3-point impedance measurement.

The substrates may be glass, SiO₂, or plastics, preferably polyethylene terephthalate, polycarbonate, or polystyrene.

Metals, for example, Au, Pt, Ag, Ti, semiconductors, for example, Si, metal oxides, especially indium-tin oxide (ITO), or conductive polymers such as polyanilines, polythiophenes, especially polyethylene dioxythiophene, polyphenylenes, polyphenylene vinylene, polythiophene vinylene, or polypyrroles, are suitable for the electrodes.

Multiplex circuits are used in order to drive a multiplicity of individual electrodes.

With an impedance measurement which operates with AC voltages or alternating currents, the solution according to the invention differs from the immediate prior art (direct-current detection) by the intrinsically available opportunity for quantifying the analytes to be detected.

Owing to the possible high packing density of the functional elements on the measurement electrode, which may also be referred to as a chip, the device according to the invention is suitable as a platform for DNA arrays and protein arrays.

### BRIEF DESCRIPTION OF THE DRAWINGS

The invention will now be described in greater detail with reference to the drawings, wherein:

FIG. 1 shows a recognition reaction on a measurement electrode

FIG. 2 shows a device for detecting DNA on ITO electrodes

FIG. 3 shows impedance spectra of a hybridization reaction

FIG. 4 shows a vertical arrangement of the electrode structure

FIG. 5 shows a vertical arrangement of electrode/insulator layer sequences

FIG. 6 shows planar electrodes in an array form

### EXAMPLES

The invention will now be described by the following non-limiting examples:

### Example 1

Method and Device for Detecting DNA on ITO Electrodes

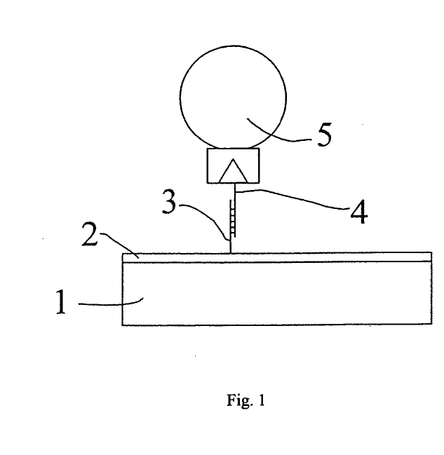

The electronic component as a platform for the recognition reaction is based on glass supports 1 coated with ITO (indium-tin oxide) (Merck, 9R1507, ohm/square: 13, ITO layer thickness: 125 nm) (FIG. 1), referred to below as chips.

Capture DNA 3 was bound to the ITO surfaces 2 as follows. 200 g of L-lysine, 50 g of caprolactam, 50 g of 1,6-diaminohexane and 0.5 g of TPP were made to react at 240° C.; water was distilled off. The resulting polyamide was diluted in the ratio 8:1 with NMP. 9 g of the polymer were reacted for silanization for 2 h under an N₂ atmosphere with 0.1 g of triethoxysilylpropyl isocyanate at RT; the silane reacted via urethane groups with the amino groups of the polyamide. Glass surfaces coated with indium-tin oxide were treated for 30 min with argon-induced plasma at standard pressure, and subsequently heated for 5 min to 80° C. A 1% strength solution of the silane-functional polyamide-urethane in a mixture of acetone/DMF/water (volume ratio 7.5:2:0.5 v/v/v) was incubated for 15 min at room temperature with the chip. After functionalization, the surfaces were washed with acetone and subsequently dried for 45 min at 110° C.

Capture DNA 3 (5′-amino—GTCCCCTACGGACAAGGCGCGT-3′) (SEQ ID NO.: 1) was dissolved in phosphate buffer pH 7.2 and incubated with 0.1M bis-sulfo-succinimidyl suberate (BS3) for 10 min at RT. The reaction was terminated by dilution with phosphate buffer. The capture DNA was purified by chromatography on a NAP-10 column (Pharmacia). The purified capture DNA was applied in volumes of, for example, 25 μl, onto the silanized surfaces, and incubated overnight at RT. The resulting DNA chips were washed with 1% strength ammonium hydroxide and water, and subsequently dried at RT. The unreacted amino groups on the chip surface were blocked by overnight incubation with 0.4 mg/ml of BS3 in 0.1M phosphate buffer pH 7.2.

DNA hybridization reactions were carried out on the chip faces coated with capture DNA, by using an analyte DNA sample 4. The match DNA analyte with the sequence 5′-biotin—TTTTTCGCGCCTTGTCCGTAGGGGACT-3′(SEQ ID NO.: 2) was used as a positive control. The complete mismatch analyte with the sequence 5′-biotin—GTCCCCTACGGACAAGGCGCGT-3′ (SEQ ID NO.: 1) was used as a negative control. 10-9M solutions of the DNAs in Tris buffer pH 8, 1M NaCl, 0.005% SDS, were incubated with the respective chip in a volume of 25 μl for 0.5 h at 56° C. Washing was then carried out with hybridization buffer, in order to remove unhybridized DNA from the chip surface. The hybridized target DNAs were incubated for 4 h at RT with a solution of streptavidin-gold 5 (diameter of the gold particles 10 nm, Sigma). The chips were washed with water and subsequently dried at RT. The gold-labeled nucleic acids were treated once for 5 min at room temperature with the enhancer solution from the company Biocell (Biocell L 15) and subsequently dried.

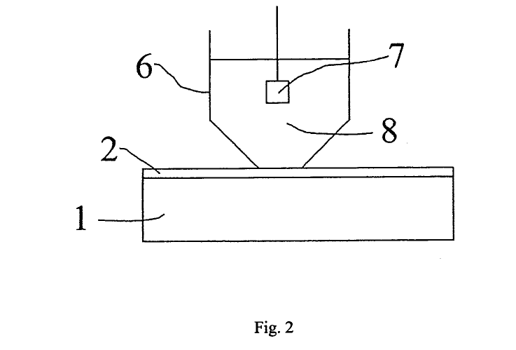

The impedimetric measurements |Z(ω)| (magnitude of the complex impedance) of the hybridization reactions were measured in 2-point geometry over a frequency range of between 0.1 Hz and 100 kHz with a predetermined AC voltage amplitude of 5 mV by using an EG&G Model 283 potentiostat/galvanostat. To that end, a open-bottomed Teflon pot 6 with a bore of 1.6 mm², which defined an electrode area of 2 mm², was placed on the chip 1 (FIG. 2). While the coated ITO electrode 2 constituted the measurement electrode, a porous tantalum electrode 7 with a total surface area of about 250 cm² was used as the counterelectrode. 0.5M NaCl was used as the electrolyte 8.

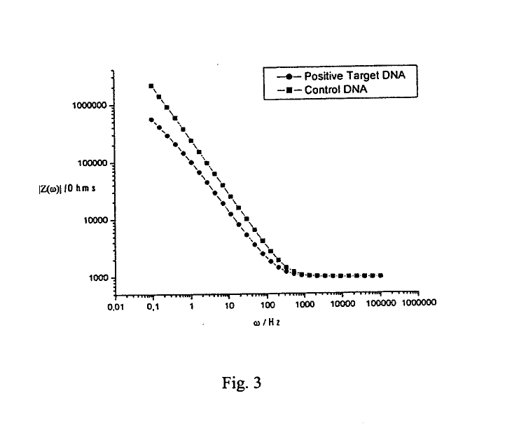

FIG. 3 shows the impedance spectra for the hybridization reaction of the capture DNA with the positive analyte DNA and the control hybridization reaction. A significant reduction in |Z(ω)| was measurable for the positive reaction.

### Example 2

Method and Device for Detecting DNA with Vertically Arranged Electrode Structures in Microchannels

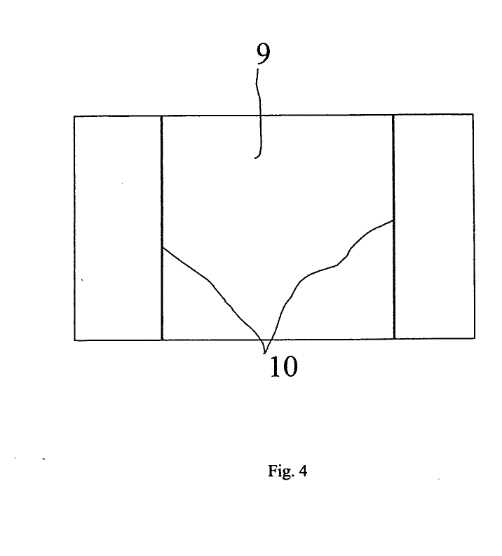

A vertical arrangement of an electrode structure according to FIG. 4 is an alternative embodiment of an electronic component according to the invention. A microchannel 9 with a width of, for example, 20 μm, is made through the layer structure by means of photolithography using ion-beam etching. A subsequent electrochemical metal deposition process leads to metallization 10 of the channel, which is therefore available with its full internal area as the measurement electrode. Immobilization and conduct of the assay take place on the inside of this microchannel in a similar fashion to Example 1.

### Example 3

Method and Device for Detecting DNA with Electrode Structures Arranged Vertically Above one Another

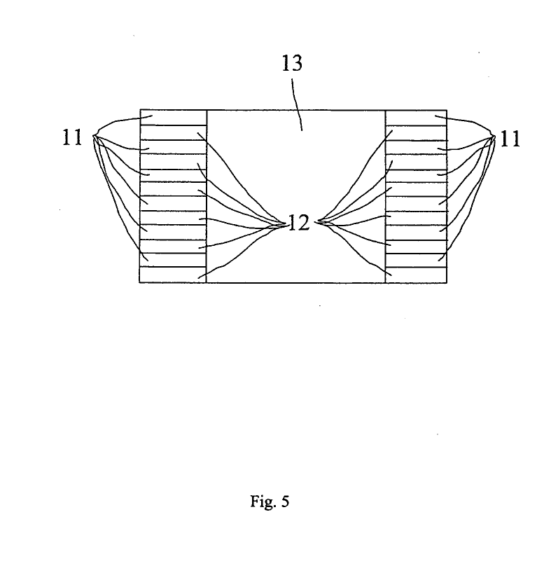

A vertical arrangement of electrode/insulator layer sequences according to FIG. 5 is an alternative embodiment of an electronic component according to the invention. Alternating layers of electrodes 11 and insulator layers 12 are deposited above one another using multistage evaporation-coating or sputtering processes. A microchannel 13 with a width of, for example, 20 μm, is made through the layer structure using ion-beam etching. Immobilization and conduct of the assay take place on the inside of this microchannel in a similar fashion to Example 1. If different capture DNAs are selectively immobilized on the various electrodes, a multiplexable microchannel for the impedimetric analysis is produced with this structure.

### Example 4

Method and Device for Detecting DNA with Planar Electrodes in a Array Form and a Multiplex Instrument

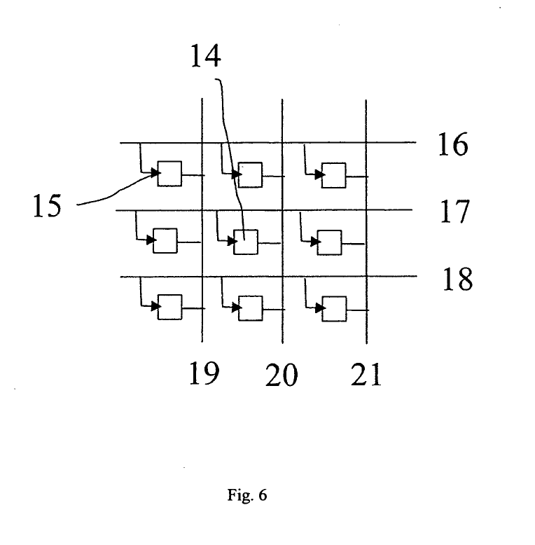

The sensor surface consists of a network of individual electronic components 14 according to Example 1 or Example 2, which are joined to one another via non-linear elements, for example diodes 15, and control lines 16-21 (FIG. 6). Immobilization and conduct of the assay take place on the inside of this microchannel in a similar fashion to Example 1. In order to read an individual component 14, the row control lines 17 are set to an on-state voltage in relation to the column control lines 20. At the same time, the row and column control line pairs 16/19, 16/20, 16/21, 17/19, 17/21, 18/19, 18/20 and 18/21 associated with the other components are set to the inverse voltage, or off-state voltage. N×N components are driven via two 2×N control lines. The electrical drives of these lines are provided by standard multiplex circuits.

It should be understood that the preceding is merely a detailed description of a few embodiments of this invention and that numerous changes to the disclosed embodiments can be made in accordance with the disclosure herein without departing from the spirit or scope of the invention. The preceding description, therefore, is not meant to limit the scope of the invention. Rather, the scope of the invention is to be determined only by the appended claims and their equivalents.

What is claimed is:

1. A method for detecting at least one analyte using a recognition reaction, said method comprising the following steps:
(a) providing a device comprising:
(i) a measurement electrode with a biofunctional surface, the biofunctional surface having recognition elements for the analyte,
(ii) one or more counterelectrodes, and
(iii) a liquid electrolyte between the measurement electrode and the one or more counterelectrodes,
(b) bringing at least one analyte labeled with an electrically active labeling unit into contact with the biofunctional surface, the electrically active labeling unit either having been bound to the analyte before the analyte is contacted with the biofunctional surface or being bound to the analyte after the analyte is contacted with the biofunctional surface,
(c) applying (i) a time-varying voltage or (ii) a time-varying current between a first counterelectrode and the measurement electrode, and
(d1) either in case (c)(i) measuring the current or in case (c)(ii) measuring the voltage between the first counterelectrode and the measurement electrode, or
(d2) in case (c)(i) measuring the current or in case (c)(ii) measuring the voltage between a second or subsequent counterelectrode and the measurement electrode.

2. Method according to claim 1, wherein the recognition elements are covalently or non-covalently immobilized on the measurement electrode.

3. Method according to claim 1, wherein the time-varying voltage is an AC voltage or a pulsed voltage.

4. Method according to claim 1, wherein the time-varying voltage is an alternating current or a pulsed current.

5. Method according to claim 1, wherein the impedance between the measurement electrode and the first or another counterelectrode is determined.

6. Method according to claim 5, wherein capacitance between the measurement electrode and the first, second or subsequent counterelectrodes is derived from the impedance measurement with the use of suitable equivalent circuit diagrams.

7. Method according to claim 1, wherein a DC voltage is superimposed on the time-varying voltage.

8. Method according to claim 1, wherein a direct current is superimposed on the time-varying current.

9. Method according to claim 1, wherein the recognition reaction constitutes an immunoassay or a DNA assay.

10. Method according to claim 9, wherein the recognition reaction constitutes an SNP assay.

11. Method according to claim 1, wherein the electrically active labeling unit has been bound to the analyte before the analyte is contacted with the biofunctional surface, and an unlabeled analyte is also brought into contact with the biofunctional surface.

12. Method according to claim 1, wherein an analyte molecule is labeled with a plurality of electrically active labeling units.

13. Method according to claim 1, wherein the electrically active labeling unit has a dielectric constant in the range of from 5 to 15,000.

14. Method according to claim 13, wherein the electrically active labeling unit has a dielectric constant in the range of between 10 and 1,500.

15. Method according to claim 1, wherein the electrically active labeling unit has a size in the range of from 1 to 100 nm.

16. Method according to claim 15, wherein the electrically active labeling unit has a size in the range of from 1 to 30 nm.

17. Method according to claim 16, wherein the electrically active labeling unit has a size in the range of from 1 to 2 nm.

18. Method according to claim 1, wherein the electrically active labeling unit is at least one of nanoparticles, metal complexes and/or clusters of conductive materials.

19. Method according to claim 18, wherein the electrically active labeling unit is at least one of Au, Ag, Pt, Pd, Cu or carbon.

20. Method according to claim 18, wherein the nanoparticles or clusters are made of titanates, materials which crystallize in a perovskite lattice, TiO₂ or lead compounds.

21. Method according to claim 18, wherein the electrically active labeling unit is at least one of carbon nanotubes, nonconductive particles with a conductive coating or nonconductive particles with a metallic coating.

22. Method according to claim 18, wherein the electrically active labeling unit is at least one of conductive polymers.

23. Method according to claim 22, wherein the conductive polymers are polyanilines, polythiophenes, polyphenylenes, polyphenylene vinylene, polythiophene vinylene, or polypyrrole.

24. Method according to claim 23, wherein the conductive polymer is polyethylene dioxythiophene.

25. Method according to claim 1, wherein the labeling unit is one of enzymes which form electrically active labeling units by the reaction of a substrate.

26. Method according to claim 25, wherein the labeling unit comprises horseradish peroxidase (HRP).

27. Method according to claim 26, wherein horseradish peroxidase (HRP) catalyses the polymerisation of a conductive polymer or catalyses the deposition of a biotinylated polymer, to whose biotins labeling units can be bound via avidin, NeutrAvidin or streptavidin.

28. Method according to claim 27, wherein the conductive polymer is polyaniline or polyethylene dioxythiophene.

29. Method according to claim 27, wherein the electrically active labeling units are autometallographically enlarged.

30. Method according to claim 29, wherein Ag or Au is used for the autometallographic enlargement.

31. A device for detecting at least one analyte using a recognition reaction, said device comprising:
(a) at least one measurement electrode with a biofunctional surface, the biofunctional surface having recognition elements for the analyte,
(b) one or more counterelectrodes,
(c) a liquid electrolyte between the measurement electrode and the counterelectrodes,
(d) at least one analyte, which is labeled with an electrically active labeling unit and is in contact with the recognition elements of the biofunctional surface,
(e) either (i) a voltage source for applying a time-varying voltage or (ii) a current source for applying a time-varying current between a first counterelectrode and the measurement electrode, and
(f) a measuring instrument for:
(i) measuring in case (e)(i) the current or in case (e)(ii) the voltage between the first counterelectrode and the measurement electrode, or
(ii) measuring in case (e)(i) the current or in case (e)(ii) the voltage between a second or subsequent counterelectrode and the measurement electrode.

32. Device according to claim 31, wherein the recognition elements are covalently or non-covalently immobilized on the measurement electrode.

33. Device according to claim 31, wherein the time-varying voltage is an AC voltage or a pulsed voltage.

34. Device according to claim 31, wherein the time-varying voltage is an alternating current or a pulsed current.

35. Device according to claim 31, wherein a DC voltage is superimposed on the time-varying voltage.

36. Device according to claim 31, wherein a direct current is superimposed on the time-varying current.

37. Device according to claim 31, wherein the electrically active labeling unit has been bound to the analyte before the analyte is contacted with the biofunctional surface, and an unlabeled analyte is also brought into contact with the biofunctional surface.

38. Device according to claim 31, wherein an analyte molecule is labeled with a plurality of electrically active labeling units.

39. Device according to claim 31, wherein the electrically active labeling unit has a dielectric constant in the range of from 5 to 15,000.

40. Device according to claim 39, wherein the electrically active labeling unit has a dielectric constant in the range of between 10 and 1,500.

41. Device according to claim 31, wherein the electrically active labeling unit has a size in the range of from 1 to 100 nm.

42. Device according to claim 41, wherein the electrically active labeling unit has a size in the range of from 1 to 30 nm.

43. Device according to claim 42, wherein the electrically active labeling unit has a size in the range of from 1 to 2 nm.

44. Device according to claim 31, wherein the electrically active labeling unit is at least one of nanoparticles, metal complexes and/or clusters of conductive materials.

45. Device according to claim 44, wherein the electrically active labeling unit is at least one of Au, Ag, Pt, Pd, Cu or carbon.

46. Device according to claim 44, wherein the nanoparticles or clusters are made of titanates, materials which crystallize in a perovskite lattice, TiO₂ or lead compounds.

47. Device according to claim 44, wherein the electrically active labeling unit is at least one of carbon nanotubes, nonconductive particles with a conductive coating or nonconductive particles with a metallic coating.

48. Device according to claim 44, wherein the electrically active labeling unit is at least one of conductive polymers.

49. Device according to claim 48, wherein the conductive polymers are polyanilines, polythiophenes, polyphenylenes, polyphenylene vinylene, polythiophene vinylene, or polypyrrole.

50. Device according to claim 49, wherein the conductive polymer is polyethylene dioxythiophene.

51. Device according to claim 31, wherein the labeling unit is one of enzymes which form electrically active labeling units by the reaction of a substrate.

52. Device according to claim 51, wherein the labeling unit comprises horseradish peroxidase (HRP).

53. Device according to claim 52, wherein horseradish peroxidase (HRP) catalyses the polymerization of a conductive polymer or catalyses the deposition of a biotinylated polymer, to whose biotins labeling units can be bound via avidin, NeutrAvidin or streptavidin.

54. Device according to claim 53, wherein the conductive polymer is polyaniline or polyethylene dioxythiophene.

55. Device according to claim 53, wherein the electrically active labeling units are autometallographically enlarged.

56. Device according to claim 55, wherein Ag or Au is used for the autometallographic enlargement.

57. Device according to claim 31, wherein the surface of the measurement electrode is divided into a plurality of conductive regions.

58. Device according to claim 57, wherein the conductive regions are of planar configuration.

59. Device according to claim 57, wherein the conductive regions have sizes in the range of from 1 to 20×1 to 20 μm².

60. Device according to claim 59, wherein the conductive regions have sizes in the range of from 5 to 15×5 to 15 μm².

61. Device according to claim 60, wherein the conductive regions have sizes of 10×10 μm².

62. Device according to claim 57, wherein one type of recognition element is immobilized in each conductive region.

63. Device according to claim 57, wherein the same type of recognition elements are immobilized in a plurality of conductive regions.

64. Device according to claim 57, wherein a plurality of conductive regions, which respectively differ in their size by a factor, are in each case used for one type of recognition unit.

65. Device according to claim 64, wherein the factor is in the range of from 5 to 15.

66. Device according to claim 65, wherein the factor is in the range of from 9 to 11.

67. Device according to claim 57, wherein the conductive regions are configured as channels in a substrate.

68. Device according to claim 57, wherein a plurality of electrodes are configured laterally next to one another or vertically above one another in the form of layer structures.

69. Device according to claim 57, wherein the conductive regions are configured in an alternating layer sequence of conductive and insulator layers as a microchannel in a substrate.

70. Device according to claim 57, wherein the counterelectrodes or counterelectrode and a reference electrode are fitted on the same substrate as the measurement electrode(s).

71. Device according to claim 70, wherein the substrate is one of glass, SiO₂, or plastic.

72. Device according to claim 71, wherein the substrate is one of polyethylene terephthalate, polycarbonate, or polystyrene.

73. Device according to claim 31, wherein the conductive regions consist of metals, semiconductors, metal oxides, or conductive polymers.

74. Device according to claim 73, wherein the conductive regions consist of Au, Pt, Ag, Ti, Si, indium-tin oxide, polyethylene dioxythiophene, polyphenylenes, polyphenylene vinylene, polythiophene vinylene, or polypyrrole.

75. Device according to claim 31, wherein a plurality of measurement electrodes form an array.

76. Device according to claim 31, which is a DNA array or a protein array.
